(19) United States
(12) Patent Application Publication
(10) Pub. No.: US 2025/0131567 A1
(43) Pub. Date: Apr. 24, 2025

(54) METHOD AND DEVICE FOR ASSOCIATING SETS OF CARDIOVASCULAR DATA

(71) Applicant: Medis Associated B.V., Leiden (NL)

(72) Inventors: Pieter Hendrik Kitslaar, Leiden (NL); Johannes Petrus JANSSEN, Leiden (NL); Hua MA, Rotterdam (NL); Johan Hendrikus Christiaan REIBER, Rotterdam (NL)

(21) Appl. No.: 18/833,975

(22) PCT Filed: Jan. 30, 2023
(86) PCT No.: PCT/NL2023/050037

§ 371 Date: Jul. 29, 2024

(30) Foreign Application Priority Data
Jan. 31, 2022 (NL) 2030789

(51) Int. Cl. G06T 7/00; A61B 5/00; A61B 8/08

(52) U.S. Cl. CPC G06T 7/0014; A61B 5/0066; A61B 8/0891; G06T 2207/10101; G06T 2207/10132; G06T 2207/30101; G06T 2207/30172

(57) ABSTRACT

There is no one-technology-fits-all data acquisition technology for body vessel, anomalies in vessel walls and per-vascular matter. Therefore, to be able to collect all acquirable data in one model or in one visualisation, data obtained using various data acquisition methods is to be combined. This is a challenge in particular if some data is collected from outside the body-CT or X-ray and other data is collected intravascular-IVUS or OCT; with IVUS and OCT, geometrical data like bends in vessels is not visible. By identifying points of interest related to particular features of a vessel, like side branches, data obtained using different acquisition methods may be aligned by associating points of interest in two or more data sets that identify the same feature over the data sets and using that as a basis for merging data to provide image data with vessel and matter around it.

### TECHNICAL FIELD

The various aspects and variations thereof relate to associating externally acquired data on a geometric representation of a vascular structure in electronic format with intravascular acquired data of the vascular structure, comprising data on matter outside and in the vicinity of the vessel, including in the vessel wall.

### BACKGROUND

Various ways of imaging allow various ways of identifying vessel anatomy, like a cardiovascular structure or a cerebrovascular structure. The vessel lumen can be imaged for example using X-ray and contrast dye injected in the vessel, using Computer Tomography (CT) scanning, using intravascular ultrasound (IVUS), using Optical Coherence Tomography (OCT) and other methods. Some of these methods are suitable for acquiring data allowing to reconstruct a three-dimensional model of the vascular structure, while others are suitable for acquiring data allowing identification of particular matter surrounding the lumen of a vessel, either in the wall of the vessel or just outside the wall of the vessel. With particular data acquisition methods, the kind of the matter may also be identified, like calcified matter, lipid matter, necrotic matter or other kinds of matter.

### SUMMARY

An issue identified is that whereas detailed data on different characteristics of the vessel structure may be obtained using different data acquisition methods, there is no one-technology-fits-all data acquisition technology. Therefore, to be able to collect all acquirable data in one model or in one visualisation, data obtained using various data acquisition methods is to be combined. This may be a challenge in particular if some data is collected from outside the body-like CT or X-ray—and other data is collected intravascular-IVUS or OCT. An issue with IVUS and OCT is that geometrical data like bends in vessels is not visible, which makes visualisation of the matter in vessel walls that has been identified, in combination with X-ray data challenging.

Thereto, a first aspect provides, in an electronic computing device, a method of associating a first set of data comprising a geometrical representation of a vessel in a body of a mammal and a human being in particular and a second set of data on the structure of the vessel, the second set of data having been acquired intravascular. The method comprises, based on the first set of data, at first positions along a length of the vessel, identifying first points of interest of the vessel at the first position and, based on the second set of data, at second positions along a length of the vessel, identifying second points of interest of the vessel at the second position. The method further comprises matching the first set of data to the second set of data, based on the first location and the second location on one hand and the first points of interest and the second points of interest on the other hand, identifying matter around the lumen based on the second set of data and obtaining at least one first matter location of the identified matter and, based on the matching, associating the first matter location of the identified matter with a second matter location in the second set of data relative to the length of the vessel. Subsequently, a third set of data is provided, the third set of data comprising a combination of the geometrical representation of the vessel and locations of the identified matter relative to the geometrical representation of the vessel.

The first set of data may be acquired using CT scanning, X-ray scanning, other, or a combination thereof. In case of use of X-ray, two X-ray images may be acquired, for example using a contrast dye, under an angle relative to one another and from those two images, a three-dimensional model of the vessel under scrutiny may be construed. In another example, only one X-ray image is provided, as a two-dimensional image. The second set of data may be acquired using intravenous ultrasound (IVUS), optical coherence tomography (OCT), other similar technologies or a combination thereof. Such data does not comprise data on geometry of a vessel in the sense that data is basically acquire relative to a centreline of the vessel along which a probe travels through the vessel. This means that for example bends or curves in a vessel cannot be detected from the acquired data.

The points of interest may differ based on methods for data acquisition used. Deviations in the lumen as to size, shape, area, other, or a combination thereof may be used as points of interests or vessel landmarks to characterise a particular position in the vessel. Additionally or alternatively, locations of branches in the vessel, matter detected in the wall of a vessel or just outside a vessel may also be used to identify points of interest. The actual points of interest used for matching the two sets of data may depend on the data acquisition methods used. The points of interest should be detectable using both data acquisition methods. For example, IVUS may be used for detecting calcified matter in a vessel wall, just as CT scanning—but this is not possible with X-ray. With X-ray, side branches are well visible, as with OCT and IVUS. And with X-ray, resolution of the lumen is better than with CT, which makes X-ray based data suitable to match with OCT or IVUS based on deviations in lumen geometry. And CT data may also be combined with X-ray data. In another implementation, also three or more sets of data may be combined using this aspect.

The two sets of data are matched by identifying in each of the sets one or more points of interest relating to the same physical feature or features of a vessel, for example a side branch or calcified matter in the vessel wall. This allows one or more first locations in the first set of data to be matched to second locations in the second set of data. Next, locations in between points of interest may be registered with both data sets.

Lastly, the data thus combined in the third set of data may be presented in a two-dimensional or three-dimensional model-depending on whether the first set of data provides two-dimensional or three-dimensional data—with the vessel itself as well as with any matter outside the lumen, either in the vessel wall or as perivascular matter.

In one example, the first points of interest are associated with first geometrical features of a first lumen of the vessel at the first position and the second points of interest are associated with second geometrical features of a second lumen of the vessel at the second position. Such geometrical features may be shape, area size, symmetry and the like as well as deviations therein from one or more reference values, side branches, bends or curves, other, or a combination thereof. Depending on data acquisition methods used, different types of geometrical features may be identified and employed.

In another example, identifying first points of interest of the first lumen comprises, based on the first set of data, along a first length of the vessel, identifying a first deviation location of a first deviation at which the lumen deviates from a pre-determined shape by more than a pre-determined first threshold; and identifying second points of interest of the second lumen comprises, based on the second set of data, along a second length of the vessel, identifying a second deviation location of a second deviation at which the lumen deviates from a pre-determined shape by more than a pre-determined second threshold; and the matching further comprises matching the first set of data to the second set of data, based on the first deviation location and the second deviation location on one hand and the first deviations and the second deviations on the other hand. Based on the data acquisition method used, particular deviations in geometrical parameters may be conveniently calculated and processed for use in matching.

A further example further comprises obtaining data on a first angular position of the first point of interest at the first location, relative to the lumen, and a second angular position of the second point of interest at the second location, relative to the lumen; in this example, the matching is further based on the first angular position and the second angular position. The angular positions relative to the lumen may be determined from a centreline of the lumen or another reference point in the lumen, the actual angle is an angle in a plane perpendicular to the centreline, relative to a reference. Such reference may be pre-determined or arbitrary, as long as it is set and stored for later use.

In yet another example, the first deviation is determined based on a first difference between a first distance reference and a first distance between a centreline of the vessel and an inner wall of the vessel at the first location; and the second deviation is determined based on a second difference between a second distance reference and a second distance between a centreline of the vessel and an inner wall of the vessel at the second location. As such, the deviation is based on an actual distance between centreline and vessel wall distance and a reference distance. The actual distance may be smaller—for example in case of stenosis—or larger—at a location of a side branch of the vessel.

In yet a further example, the first point of interest is determined at the first location at a first angular position relative to the centreline; and the second point of interest is determined at the second location at a second angular position relative to the centreline. With angular positions of points of interest known for both sets of data, the data may be matched to provide improved matching, in particular in a three-dimensional view. In particular, this allows for providing the appropriate angular location of the identified matter relative to other data with specific angular positions relative to the vessel, like bends, curves and side branches.

In again another example, the first point of interest is determined by determining a deviation at the first location at which a first value of a first parameter comprised by data in the first set of data differs from a first pre-determined value by more than a first threshold value; and the second point of interest is determined by determining a deviation at the second location at which a second value of a second parameter comprised by data in the second set of data differs from a second pre-determined value by more than a second threshold value. This allows to set particular margins for a deviation. Pre-determined may be understood as determined at the moment the method is designed. Alternatively or additionally, pre-determined may be understood as being determined before determining the difference value. as such, the pre-determined threshold may also be determined during execution of the method, yet before the difference is calculated.

In another example, the first threshold value is at least one of a fixed value and a relative value dependent on at least one of the first value, the first pre-determined value and the first threshold value; and the second threshold value is at least one of a fixed value and a relative value dependent on at least one of the second value, the second pre-determined value and the second threshold value. This allows for dedicated evaluation of data per subject.

In a further example, determining the first point of interest comprises identifying a first branch in the vessel; and determining the second point of interest comprises identifying the first branch in the vessel. Branches in vessels may be relatively simply detected, irrespective of the data acquisition method used. Hence, branches are a proper example to be used as points of interests or landmarks of a vessel. Alternatively or additionally, stents may be used as landmarks.

In another example, the identifying of matter comprises identifying of matter in a wall of the vessel. With IVUS and CT, calcified matter may be detected in a vessel wall. With IVUS, calcified matter is visible because of the shadow it provides and in common CT visualisations, calcified matter in vessel walls is shown in white,

In yet another example, the data of the second set is obtained using at least one of intravascular ultrasound and optical coherence tomography. Such data acquisition methods are known and may even be used together using a combined probe. The data of both data acquisition methods may subsequently even be combined, with geometric data.

Yet a further example further comprises displaying at least part of the third set of data by displaying at least part of the geometrical, preferably three-dimensional representation of the vessel and the identified matter relative to the vessel—but it may be two-dimensional as well. Such visualisation provides a medical practitioner to assess a state of the vessel and any anomalies that may give rise to a medical issue. As such, such representation may provide additional aid to a medical practitioner to set a diagnosis.

Again another examples comprises at the first location, obtaining a multitude of first distances at a multitude of angular locations relative to the centreline; at the second location, obtaining a multitude of second distances at a multitude of angular locations relative to the centreline; determining the first deviation at the first location if a statistical parameter of value of the multitude of first distances matches a pre-determined first statistical condition; and determining the second deviation at the second location if a statistical parameter of value of the multitude of second distances matches a pre-determined second statistical condition. This example provides a practical implementation of determining deviations in a lumen structure.

In yet another example, the first statistical parameter is based on at least one of an average, median, standard deviation, maximum and minimum of the multitude of first distances; and the second statistical parameter is based on at least one of an average, median, standard deviation, maximum and minimum of the multitude of second distances. This example provides a practical implementation of determining deviations in a lumen structure.

In a further example, the first statistical criterion is met if a pre-determined amount of first distances differs more than a first statistical threshold value from the first statistical parameter; and the second statistical criterion is met if a pre-determined amount of second distances differs more than a second statistical threshold value from the second statistical parameter. This example provides a practical implementation of determining deviations in a lumen structure.

In again another example, the first statistical criterion is met if at least one value of the first distances differs from the first statistical parameter by more than a third statistical parameter threshold value; and the second statistical criterion is met if at least one value of the second distances differs from the second statistical parameter by more than a fourth statistical parameter threshold value. This example provides a practical implementation of determining deviations in a lumen structure.

[repetition of claim 1 to follow, with explanation and advantages]

### BRIEF DESCRIPTION OF THE DRAWINGS

The various aspects and variations thereof will now be further elucidated in conjunction with drawings. In the drawings:

FIG. 1: shows an example of a device in accordance with the second aspect;

FIG. 2: shows a flowchart;

FIG. 3A: shows a three-dimensional geometric representation of a cardiovascular structure;

FIG. 3B: shows a visualisation of data obtained by means of an intravascular data acquisition method;

FIG. 4A: shows a first cross-section of a coronary artery;

FIG. 4B: shows a second cross-section of a coronary artery; and

FIG. 5: shows a visualisation of the three-dimensional geometric representation of a cardiovascular structure with lipid matter.

### DETAILED DESCRIPTION

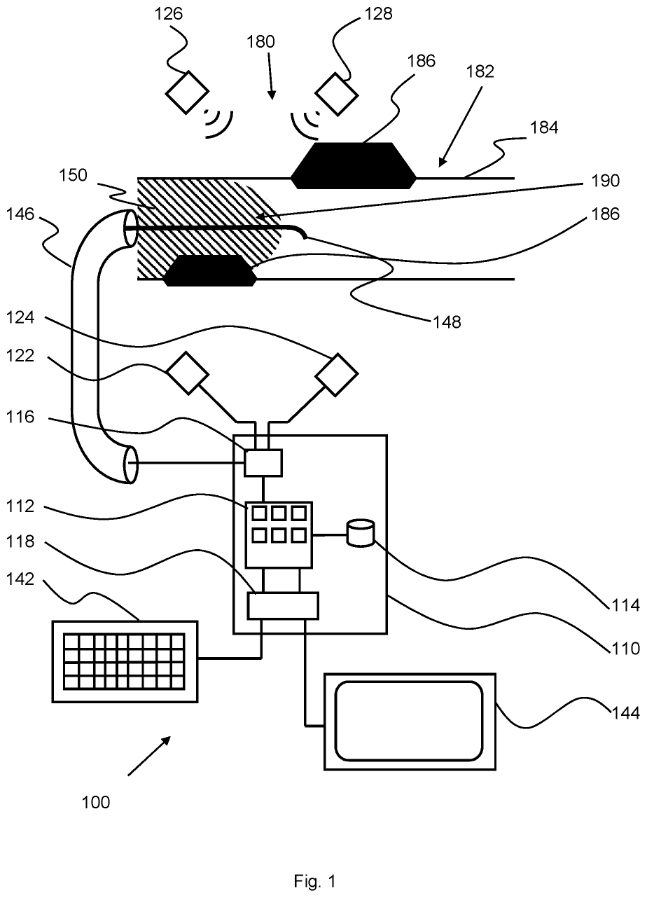

FIG. 1 shows an electronic medical data acquisition and processing system 100 as an example of the second aspect. The system 100 or parts thereof may be found in a cardiac catheterisation laboratory of a clinic or a hospital. The system 100 comprises an X-ray image acquisition module comprising a first X-ray source 126 and a second X-ray source 128, a first X-ray detector 122 arranged to receive X-ray data from the first X-ray source 126 and a second X-ray sensor 124 arranged to receive X-ray data from the second X-ray source 128. The first X-ray source 126, the second X-ray sensor 124, the first X-ray detector 122 and the second X-ray sensor 124 are arranged to obtain images of a cardiovascular structure 180 under an angle relative to one another. The angle is preferably between 25° and 45°, more preferably between 30° and 40°.

The first X-ray detector 122 and the second X-ray detector 124 are connected to data acquisition module 116 of an electronic computing device 110. The electronic computing device further comprises a processing unit 112, a storage module 114 and a peripherals I/O controller 118. The processing unit 112, which may be implemented as a microprocessor, microcontroller or other electronic data processing device, is arranged to control the various part of the electronic computing device 110 and the system 100 and arranged to execute the method according to the first aspect and implementations thereof.

The storage module 114 is arranged for storing data thereon, for example acquired by the computing device 110 from the various other parts of the system 100, either directly or after processing by the processing unit 112. The storage unit 114, as at least partially implemented as a non-transitional storage medium, is further arranged for storing computer executable code which allow the processing unit 112 to execute the method according to the first aspect and implementations thereof.

The system 100 comprises a data acquisition probe 148 for obtaining data on a wall 184 of a coronary artery 182 of the cardiovascular structure 180 as an example of a coronary vessel or blood vessel in general. The data acquisition probe 148 may be inserted in the coronary artery 182 via a catheter 146 inserted in a body of a mammal, like a human being. Additionally, the tip of the coronary catheter 146, placed into the ostium of the coronary artery 182 under scrutiny. The data acquisition probe 148 is in this variation an ultrasound sounding probe, arranged to obtain data on an internal structure of the coronary artery 182 as well as on the wall 184.

FIG. 1 shows the wall 184 comprising lipid matter 186 at multiple location in the coronary artery 182, as detected by means of the intravascular ultrasound (IVUS) imaging method. In another case or at another location in the same case, the lipid matter 186 is present outside the coronary artery 182, adjacent to an outside of the vessel wall 184 as perivascular adipose tissue (PVAT). Hence, the lipid matter 186 is present around the lumen of the coronary artery, either in the wall 184 of the artery or just outside the wall 184. Alternatively or additionally, the data acquisition probe 148 may be arranged to obtain data using optical coherence tomography. Furthermore, the catheter 146 may be used to insert contrast dye 150—or another dye such as saline—in the coronary artery 182 or another vessel of a body, which allows the coronary artery 182 to be made visible using the X-ray detectors.

The peripherals I/O controller 118 is arranged to connect the computing device 110 and the various components thereof to input device like a keyboard 142 or a touch screen for receiving data like user input. The peripherals I/O controller 118 is arranged to connect the computing device 110 and the various components thereof to output devices like an electronic display 144 and other output devices arranged to provide a user with data on processed or unprocessed data received by the computing device 110.

As shown in FIG. 1, the catheter 146 and the data acquisition probe 148 are inserted in the coronary artery 182. In the coronary artery 182, a narrowing 190 may be present. The narrowing 190 may be caused by a plaque, which may consist of various substances, such as calcium, lipid matter or any other plaque component 186. This stenotic area results in narrowing in the coronary artery 182, which, in turn results in pressure drops at the stenotic area. The narrowing 190 may result in an asymmetric or in any case not circular or elliptical cross-section of the coronary artery 182. The cardiovascular structure 180 shown by FIG. 1 may be a hypothetical structure and is not necessarily a representation of an actual anatomical structure.

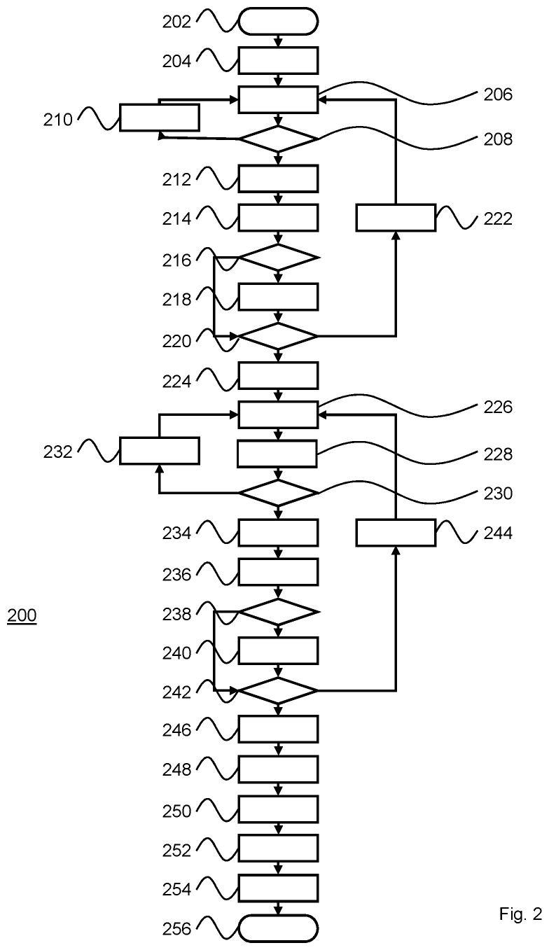

The further functionality of the system 100 and parts thereof discussed above will be further elucidated in conjunction with a flowchart 200 depicted by FIG. 2. The procedure depicted by the flowchart 200 is executed by the system 100 and the electronic computing device 110 in particular, controlled by the processing unit 112. To provide this functionality, the processing unit 112 may be programmed by means of a computer programme product comprising computer executable code. The computer programme product may be stored on the storage unit 114 as an electronic memory, which may be a non-transitory memory. The various parts of the flowchart 200 are briefly summarised below. Various steps may be swapped in order of execution or be executed in parallel, unless explicitly indicated otherwise.

| | |
|---|---|
| 202 | start of the procedure |
| 204 | obtain three-dimensional vessel structure data |
| 206 | determine lumen radius as angular |
| | position at a location along centreline |
| 208 | all positions done? |
| 210 | proceed to next angular position |
| 212 | determine lumen area |
| 214 | process radii and area data |
| 216 | any deviations of processed data? |
| 218 | register deviation data and location |
| 220 | all locations done? |
| 222 | proceed to next location |
| 224 | obtain intravascular obtained data |
| 226 | determine lumen radius as angular |
| | position at a location along centreline |
| 228 | determine matter in vessel wall at angular |
| | position and store data |
| 230 | all positions done? |
| 232 | proceed to next angular position |
| 234 | determine lumen area |
| 236 | process radii and area data |
| 238 | any deviations of processed data? |
| 240 | register deviation data and location |
| 242 | all locations done? |
| 244 | proceed to next location |
| 246 | match deviation data of the data sets |
| 248 | associate locations three-dimensional |
| | vessel structure data and IV data |
| 250 | associate determined matter data to |
| | three-dimensional vessel structure data |
| 252 | combine matter data and three-dimensional |
| | vessel structure data |
| 254 | display combined data |
| 256 | end procedure |

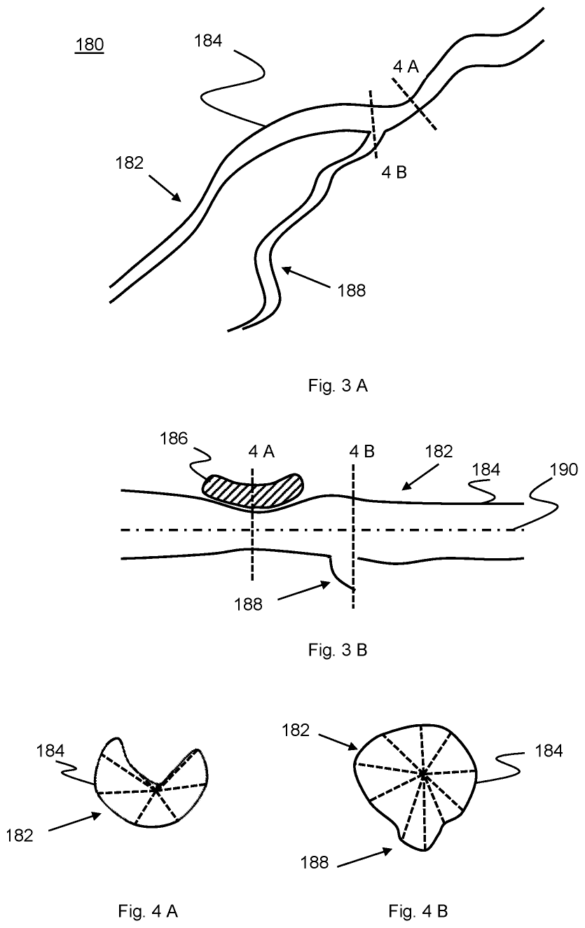

The procedure starts in a terminator 202 and proceeds to step 204 in which data on a three-dimensional vessel structure of the cardiovascular structure 180 is obtained. This data may be obtained using the system 100 (FIG. 1) or in another way, for example using computer tomography (CT) scanning. FIG. 3A depicts a three-dimensional representation of the cardiovascular structure 180, with the coronary artery 182 having the vessel wall 184 and a branch vessel 188. In step 206, at a first location within the coronary artery 182, a radius of the lumen at the first location is determined. Prior to this determination, a centreline may be determined for the coronary artery 182 as described by the three dimensional model within the obtained data set. The centreline thus determined may be used for determining radii, starting from the centreline and reaching to the inner wall 184 of the coronary artery 182.

FIG. 4A shows a cross-section of the coronary artery 184 at the location denoted “4 A” in FIG. 3A and FIG. 3B and FIG. 4B shows a cross-section of the coronary artery 184 at the location denoted “4 B” in

FIG. 3A and FIG. 3B. Hence, FIG. 4A shows a cross-section of the coronary artery 184 at a different location than FIG. 4B does. As can be seen in both FIG. 4A and FIG. 4B, the radius of the coronary artery 182 may be different at different angular positions. FIG. 4A shows a cross-section at a stenotic location of the coronary artery 182. FIG. 4B shows a cross-section at a location where the branch vessel 188 branches from the coronary artery 182.

Generally, the cross-section of a coronary artery has a substantially circular cross-section. In such cases, a coronary artery has more or less a rotation symmetrical shape. The radius of the cross-section may be different at different angular positions at the centreline, but the curvature of the vessel inner wall 184 or the lumen is, viewed from the centreline, generally concave. Furthermore, the radii at different angular positions vary, but gradually and within a particular boundary.

On the other hand, in some cases, the curvature of the lumen may be, from the centreline have a convex case. This is in particular the case if a large anomaly is present in a well of a vessel. In such case, but also in other cases, the radii of the lumen may be very different at different angular positions and may not necessarily change gradually, but rather abruptly.

As can been seen in FIG. 4A and FIG. 4B, in case of abnormalities, like stenosis and branches, the cross-section may have different shapes that are not rotation symmetrical. Furthermore, there may be significant variations of the radii at different angular positions with the centreline as starting point.

As discussed above, in step 206, a radius of the lumen of the coronary artery 182 is determined, at a particular location along the length of the coronary artery 182 and along the centreline in particular. Once that has been done, the data is stored and the procedure continues to step 208, in which is checked whether all angular positions have been checked for determining the radius at that particular angular position. The angular positions may be equidistantly distributed, for example 36 measurements may be taken at intervals of 10°, or 10 measurements may be taken at intervals of 36°—or any other number of measurements may be taken. If not all positions have been processed, the procedure branches back to step 206 via step 210 in which the next angular position is selected.

If all angular positions have been processed with respect to the value of the radius at each position for one location, the lumen area is determined in step 212 for the active location in the coronary artery 182. Next, in step 214, the data on the determined radii and area at one location is processed. The processing may comprise calculating statistical parameters like mean, median, standard deviation, minimum, maximum, other, or any combination thereof, determining outliers and removing outlier values, correction for measurement inaccuracies like noise or offset, other processing, or any combination thereof.

In step 216 is checked whether there is any deviation in the cross-section. More in particular, in step 216 may be checked whether the values of the radii are all within a particular boundary having either a pre-determined fixed value, being based on the values of the radii, being based on values resulting out of the processing of step 214, other or any combination thereof. For example, if one value of a radius varies from the median or mean by more than 10% of the median or median value or by more than a multitude-being more or less than 1—of the standard deviation, it may be determined that the cross-section is a deviating cross-section.

Root causes for variations in cross-sections and deviations in cross-sections may be numerous. An obvious cause for a deviations in cross-sectional dimensions is a side-branch. Other causes may be calcified areas, with calcified matter in the vessel wall. Also other matter in the vessel wall, like lipid matter, may result in cross-sectional variations. Hence, side branches may also be considered as deviations in the lumen.

The presence of a side branch and/or the location thereof does not have to be determined using the processing discussed above; it may, additionally or alternatively, also be detected as a geometrical lumen feature using for example image recognition. Apart from presence and location of a side branch, also presence and location of other landmark features of a coronary vessel—or other vessel under scrutiny—may be detected or otherwise identified as a geometrical lumen feature and as such, be used for co-registration of data as discussed below.

If the cross-section is determined to be deviating, the location of the cross-section is registered in step 218. With the registration, further data may be registered like area of the lumen, position along the centreline of the coronary artery 184, values of radii at various angular position, other, or a combination thereof. Subsequently, the procedure continues to step 220. If the cross-section is not determined to be deviating, the procedure continues direction to step 220 from step 216.

In step 220 is checked whether all locations along the length of the coronary artery 184 have been processed with respect to determining radii and area and with respect to determining deviations. The number of locations may be determined based on required accuracy and may be determined as an amount of locations per millimetre or centimetre or other measure of length. Additionally or alternatively, the number of locations may be set and the distance between locations may be set based on the number of locations and the length of the coronary artery 182. If not all locations have been processed, the procedure branches back to step 206 via step 222, in which the next location is selected. If all locations have been processed, the process proceeds to step 224.

In step 224, data is obtained on the coronary artery 184 that has been collected intravascular, as discussed above. The data set comprising the intravascularly obtained data does not comprise the three-dimensional vessel structure as depicted by FIG. 3A, i.e. how the vessel is curved along its length. Rather, by the nature of the process of acquiring the data, data acquired on the vessel is provided along a line over which the data acquisition probe 148 has been pulled or pushed through the coronary artery 182. This is depicted in FIG. 3B. FIG. 3B shows a centreline 190 that indicates a centre of the coronary artery 182.

The centreline 190 may be determined in various ways, for example a centre may be determined in multiple subsequent cross-sections of the coronary artery 182 and the centreline may be determined as a line that follows these centres. Alternatively, a moving average of the centre is determined based on two, three, five, ten, twenty or another multitude of cross-sections. This applies to the data shown by FIG. 3A as well. Various methods are known in the art for determining a centreline of a vessel, which may all be employed.

In step 226, a radius of the lumen of the coronary artery 184 is determined based on intravascular acquired data, at a particular angular position. This may be done as discussed above in step 206. Subsequently, in step 228, data that has been acquired intravascularly is processed to determine whether particular matter is present in the wall of the coronary artery 182. Such matter may be lipid matter, calcified matter, necrotic tissue, other, or a combination thereof.

Of such matter, the distance from the centreline with respect to smallest distance to the centreline 190 is determined, as well as largest distance to the centreline 190. With such data being available for multiple angular positions, at multiple locations along the length of the coronary artery 184 and along the centreline 190 in particular, the size and shape of the lipid matter 186 may be reconstructed, relative to the centreline, relative to the inner wall 184, relative to another feature of the coronary artery 182 or a combination thereof.

In step 230 is determined whether all angular positions along the centreline have been covered for determining the radius and any matter outside vessel lumen, either in the wall of the vessel or outside thereof. If not all positions have been covered, the process branches back to step 226 via step 232 in which the next angular position is selected. If all angular positions have been covered for determining the radius, the procedure continues to step 234.

In step 234, the lumen area is determined based on the intravascular obtained data, analogous to the action in step 214. Subsequently, in step 236, the radius data, the area data and the detected matter data is processed. The processing may be as discussed in conjunction with step 214. Subsequently, in step 238, based on the raw radius and area data, based on results of the processing, based on other data like pre-determined threshold values, it is determined whether the applicable cross-section is a deviating cross-section.

With a large variation in radii, for example the cross-section as depicted by FIG. 4A may be characterised as deviating. In this example, the deviating shape of the lumen may be caused by matter in the wall of the coronary vessel 182. It is noted that the cross-section as depicted by FIG. 4 A is not rotation symmetrical. Also such feature may be employed to determine whether the cross-section is deviating for one or more of these reasons. Also the cross-section as depicted by FIG. 4B, at a location of the branch vessel 118, may be considered to be deviating. Different criteria may be employed, such as a sudden increase in volume-due to the branching—or a large variation in values of the radii. If a cross-section at a location is considered to be deviating, data is stored in step 240 analogous to action in step 218. The process continues to step 242.

If there is no deviation determined, the procedure continues directly to step 242. In step 242 is checked whether all locations along the centreline 190 have been covered, analogous to step 220. If not all locations have been assessed, the procedure branches back to step 226, while selecting the next location along the centreline, analogous to step 222. If all locations have been processed, the procedure continues to step 246.

In step 246, deviation data acquired as discussed above, for the three-dimensional geometry data and for the intravascularly acquired data, is matched. The matching is in this variation done by assessing in particular geometry features of cross-sections that are found to be deviating. In one variation, radii at multiple positions at a particular location of a deviating cross-section obtained at a deviating location of the first data set—the geometrical three-dimensional representation—are compared to radii at multiple positions at locations to be deviating in the second data set—with intravascularly obtained data.

If the pattern of radii at locations in two sets are found to differ by less than a particular threshold, the locations in both sets may be held to be matching. It will be apparent that this step may executed as well for data at all locations, but by matching only deviating locations, processing effort is reduced. Second, matching of substantially rotation symmetrical and elliptical or circulator cross-sections for specific locations is generally more complex than with cross-sections that have abnormalities.

With sufficient processing power and appropriate pattern recognition and data comparison algorithms available, it is, however, possible to match data of the set with data on the three-dimensional representation of the cardiovascular structure 180 with the data of the set with data obtained intravascularly. With recent developments in artificial intelligence, technology is available to match sets using all or at least most data of both sets.

With matching cross-sections identified in both data sets, data in the first set and data in the second set may be associated in step 248. In particular with angular data per location available for both sets, not only the locations of both sets may be associated, but also location of particular data in one set may be associated with data of another set. For example, as discussed above, an angular position of the lipid matter 186 relative to the centreline 190 may be determined based on the intravascularly acquired data.

By associating a shape of a cross-section or radii relative to an angular position to the centreline, data in both sets may also be associated with respect to angular position. In this way, it is possible to determine a location of the lipid matter 186 in the three-dimensional geometrical representation of the cardiovascular structure 180 and in particular relative to the length of the coronary artery 182 and the centreline thereof in particular, but it is also possible to reconstruct an angular position of the lipid matter 186 relative to the coronary artery 182.

The reconstruction may be performed in step 250. In step 252, based on the associating, data of both data sets may be combined in step 254. In particular, data on the lipid matter 186 in the wall of the coronary vessel 182 available in the second dataset may in this way be combined with data in the first dataset providing a three-dimensional representation of the cardiovascular structure 180.

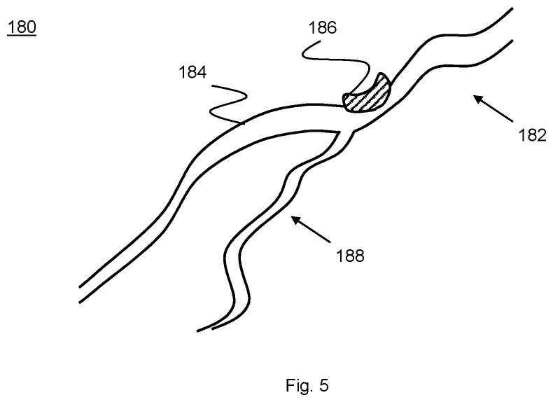

A result is shown in FIG. 5, depicting the three-dimensional representation of the cardiovascular structure 180, with the lipid matter 186 at the actual longitudinal and radial position relative to the coronary artery 182. This structure may be displayed in step 254 in various ways. FIG. 5 shows a three-dimensional display of the merged data. Alternatively or additionally, a two-dimensional view may be provided with a two-dimensional geometrical view of the cardiovascular structure 180, with for example the lipid matter 186 or other matter like calcified matter, at the actual longitudinal and/or radial position relative to the coronary artery 182. Alternatively or additionally, a cross-sectional view of a vessel may be provided, with the total vessel wall with any matter present therein, and with, in case selected and present, perivascular matter. After display, the procedure ends in a terminator 256.

In summary, there is no one-technology-fits-all data acquisition technology for body vessel, anomalies in vessel walls and per-vascular matter. Therefore, to be able to collect all acquirable data in one model or in one visualisation, data obtained using various data acquisition methods is to be combined. This is a challenge in particular if some data is collected from outside the body-CT or X-ray—and other data is collected intravascular-IVUS or OCT; with IVUS and OCT, geometrical data like bends in vessels is not visible. By identifying points of interest related to particular features of a vessel, like side branches, data obtained using different acquisition methods may be aligned by associating points of interest in two or more data sets that identify the same feature over the data sets and using that as a basis for merging data to provide image data with vessel and matter around it.

1. In an electronic computing device, for execution or executed by the electronic computing device, a method of associating a first set of data comprising a geometrical representation of a vessel in a body of a mammal and a second set of data on the structure of the vessel, the second set of data having been acquired intravascular, the method comprising:
identifying first points of interest of the vessel at first locations along a length of the vessel, based on the first set of data;
identifying second points of interest of the vessel at second locations along a length of the vessel, based on the second set of data;
matching the first set of data to the second set of data, based on the first locations and the second locations on one hand and the first points of interest and the second points of interest on the other hand;
identifying matter around the lumen of the vessel based on the second set of data and obtaining at least one first matter location of the identified matter in the first set of data;
associating, based on the matching, the first matter location of the identified matter in the first set of data with a second matter location in the second set of data relative to the length of the vessel; and
providing a third set of data comprising a combination of the geometrical representation of the vessel and locations of the identified matter relative to the geometrical representation of the vessel.

2. The method of claim 1, wherein the first points of interest are associated with first geometrical features of one or more first lumen of the vessel at the first location and the second points of interest are associated with second geometrical features of one or more second lumen of the vessel at the second location.

3. The method of claim 1, wherein:
identifying first points of interest of the first lumen comprises, based on the first set of data, along a first length of the vessel, identifying a first deviation location of a first deviation at which the lumen deviates from a pre-determined shape by more than a pre-determined first threshold; and
identifying second points of interest of the second lumen comprises, based on the second set of data, along a second length of the vessel, identifying a second deviation location of a second deviation at which the lumen deviates from a pre-determined shape by more than a pre-determined second threshold; and
the matching further comprises matching the first set of data to the second set of data, based on the first deviation location and the second deviation location on one hand and the first deviations and the second deviations on the other hand.

4. The method of claim 1, further comprising:
obtaining data on a first angular position of the first point of interest at the first location, relative to the lumen, and a second angular position of the second point of interest at the second location, relative to the lumen; and
wherein the matching is further based on the first angular position and the second angular position.

5. The method of claim 3,
wherein:
the first deviation is determined based on a first difference between a first distance between a centreline of the vessel and an inner wall of the vessel at the first location; and
the second deviation is determined based on a second difference between a second distance between a centreline of the vessel and an inner wall of the vessel at the second location.

6. The method of claim 1, wherein:
the first point of interest is determined at the first location at a first angular position relative to the centreline; and
the second point of interest is determined at the second location at a second angular position relative to the centreline.

7. The method of claim 1, wherein:
the first point of interest is determined by determining a deviation at the first location at which a first value of a first parameter comprised by data in the first set of data differs from a first pre-determined value by more than a first threshold value; and
the second point of interest is determined by determining a deviation at the second location at which a second value of a second parameter comprised by data in the second set of data differs from a second pre-determined value by more than a second threshold value.

8. The method of claim 7, wherein:
the first threshold value is at least one of a fixed value and a relative value dependent on at least one of the first value, the first pre-determined value and the first threshold value; and
the second threshold value is at least one of a fixed value and a relative value dependent on at least one of the second value, the second pre-determined value and the second threshold value.

9. The method of claim 1, wherein:
determining the first point of interest comprises identifying a first branch in the vessel; and
determining the second point of interest comprises identifying the first branch in the vessel.

10. The method of claim 1, wherein the identifying of matter comprises identifying of matter in a wall of the vessel.

11. The method of claim 1, wherein the data of the second set of is obtained using at least one of intravascular ultrasound and optical coherence tomography.

12. The method of claim 1,
further comprising displaying at least part of the third set of data by displaying at least part of the three-dimensional representation of the vessel and the identified matter relative to the vessel.

13. The method of claim 5, wherein
the first point of interest is determined at the first location at a first angular position relative to the centreline; and
the second point of interest is determined at the second location at a second angular position relative to the centreline;
the method further comprising:
at the first location, obtaining a multitude of first distances at a multitude of angular locations relative to the centreline;
at the second location, obtaining a multitude of second distances at a multitude of angular locations relative to the centreline;
determining the first deviation at the first location if a statistical parameter of value of the multitude of first distances matches a pre-determined first statistical condition; and
determining the second deviation at the second location if a statistical parameter value of the multitude of second distances matches a pre-determined second statistical condition.

14. The method of claim 13, wherein:
the first statistical parameter is based on at least one of an average, median, standard deviation, maximum and minimum of the multitude of first distances; and
the second statistical parameter is based on at least one of an average, median, standard deviation, maximum and minimum of the multitude of second distances.

15. The method of claim 14, wherein:
the first statistical criterion is met if a pre-determined amount of first distances differs more than a first statistical threshold value from the first statistical parameter; and
the second statistical criterion is met if a pre-determined amount of second distances differs more than a second statistical threshold value from the second statistical parameter.

16. The method of claim 15, wherein:
the first statistical criterion is met if at least one value of the first distances differs from the first statistical parameter by more than a third statistical parameter threshold value; and
the second statistical criterion is met if at least one value of the second distances differs from the second statistical parameter by more than a fourth statistical parameter threshold value.

17. A device arranged for associating a first set of data comprising a geometrical representation of a vessel in a body of a mammal with a second set of data on the structure of the vessel, the second set of data having been acquired intravascularly, the device comprising a processing unit arranged to:
based on the first set of data, at first positions along a length of the vessel, identify first points of interest of the vessel at first locations;
based on the second set of data, at second positions along a length of the vessel, identify second points of interest of the vessel at second locations;
match the first set of data to the second set of data, based on the first locations and the second locations on one hand and the first points of interest and the points of interest on the other hand;
identify matter around the lumen based on the second set of data and obtain at least one first matter location of the identified matter;
based on the matching, associate the first matter location of the identified matter with a second matter location in the second set of data relative to the length of the vessel; and
provide a third set of data comprising a combination of the geometrical representation of the vessel and locations of the identified matter relative to the geometrical representation of the vessel.

18. A computer programme product comprising computer executable code arranged to cause a processing unit of a processing device, when programmed in accordance with the computer executable code cause the processing unit to perform a method of associating a first set of data comprising a geometrical representation of a vessel in a body of a mammal with a second set of data on the structure of the vessel, the second set of data having been acquired intravascularly, the method comprising:
based on the first set of data, at first positions along a length of the vessel, identifying first points of interest of the vessel at first locations;
based on the second set of data, at second positions along a length of the vessel, identifying second points of interest of the vessel at second locations;
matching the first set of data to the second set of data, based on the first locations and the second locations on one hand and the first points of interest and the second points of interest on the other hand;
identifying matter around the lumen based on the second set of data and obtaining at least one first matter location of the identified matter;
based on the matching, associating the first matter location of the identified matter with a second matter location in the second set of data relative to the length of the vessel; and
providing a third set of data comprising a combination of the geometrical representation of the vessel and locations of the identified matter relative to the geometrical representation of the vessel.

19. A non-transitory computer readable medium comprising computer executable code for causing a processor to perform a method of associating a first set of data comprising a geometrical representation of a vessel in a body of a mammal and a second set of data on the structure of the vessel, the second set of data having been acquired intravascularly, the method comprising:
based on the first set of data, at first positions along a length of the vessel, identifying first points of interest of the vessel at first locations;
based on the second set of data, at second positions along a length of the vessel, identifying second points of interest of the vessel at second locations;
matching the first set of data to the second set of data, based on the first locations and the second locations on one hand and the first points of interest and the second points of interest on the other hand;
identifying matter around the lumen based on the second set of data and obtaining at least one first matter location of the identified matter;
based on the matching, associating the first matter location of the identified matter with a second matter location in the second set of data relative to the length of the vessel; and
providing a third set of data comprising a combination of the geometrical representation of the vessel and locations of the identified matter relative to the geometrical representation of the vessel.

20. The device of claim 17, wherein the geometrical representation of the vessel is a three-dimensional representation.
